(19) United States
(12) Patent Application Publication
(10) Pub. No.: US 2005/0054556 A1
(43) Pub. Date: Mar. 10, 2005

(54) Antiphlogistic agent and the use thereof

(75) Inventor: Martin Gorne, Hamburg (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: KOSMAS KG, Norderstedt (DE)

(21) Appl. No.: 10/496,692

(22) PCT Filed: Nov. 27, 2002
(86) PCT No.: PCT/EP02/13385

(30) Foreign Application Priority Data
Nov. 27, 2001 (DE) 101 58 039.8

(51) Int. Cl. A61K 38/16; C12P 21/06

(52) U.S. Cl. 514/2; 435/68.1

(57) ABSTRACT

The present invention relates to pharmaceutical compositions based on antiinflammatory active ingredients and to the use thereof for the treatment of rheumatoid diseases. The compositions comprise lactalbumin hydrolysate or a fraction thereof. Substantially lower doses of the antiinflammatory drugs are thus possible than is conventionally necessary to achieve a particular antiinflammatory effect.

The present invention relates to pharmaceutical compositions based on antiinflammatory active ingredients and to the use thereof for the treatment of rheumatoid diseases.

Lactalbumin hydrolyzates have been used for some time as dietary foods. For example, a peptide mixture composed of lactalbumin, meat and soy is used in cases of maldigestion, malabsorption, in consuming processes and for strengthening. Lactalbumin hydrolyzates are distinguished by being able to be absorbed very readily and being available for metabolism in a very short time. The low molecular weight fraction in particular is particularly suitable for absorption and further metabolic utilization.

In addition, there have also been reports that degradation products said to have pharmacological effects can be obtained starting from whey protein, lactalbumin, α-lactalbumin, lactoferrin, β-lactoglobulin, lysozyme or serum albumin. Thus, DE-A 39 22 453 and PCT/EP86/00016 (WO 86/04217) for example mention an analgesic, antiinflammatory or antimutagenic activity. It is therefore recommended that the degradation products be used for the treatment of painful inflammations of all types, neurodermatitis, arthritis, rheumatism, but also of glaucomas. The milk constituents obtainable according to DE 38 29 552 A1 are also said to have characteristic pharmacological properties and to be usable for the treatment of neurodermatitis, allergies, glaucoma and for immunostimulation. However, there is vigorous controversy about the pharmacological activity of these lactalbumin hydrolyzates. Nor has it been possible to demonstrate it in the manner necessary for clinical use.

In the treatment of chronic inflammatory diseases such as, for example, certain rheumatoid diseases with steroidal and non-steroidal antiinflammatory drugs, it is desirable for the doses of active ingredient to be as low as possible in order to reduce the side effects which are associated with the long-term treatment, which is usually necessary, to a tolerable level. For example, iatrogenically induced Cushing's syndrome may occur during corticoid therapy, or duodenal and gastric ulcers may develop. In respect of non-steroidal antiinflammatory drugs, owing to the heterogeneity of the underlying classes of substances, a large number of adverse effects is known. For example, phenylbutazones may cause agranulocytosis or edemas, acetylsalicylic acid, arylacetic acid derivatives and arylpropionic acid derivatives may cause gastric bleeding, pyrazolones may cause cytopenia, anthranilic acid derivatives may cause glumerulonephritis, azapropazone may cause renal dysfunctions, piroxicam and tenoxican may cause renal and intestinal dysfunctions, paracetamol may cause liver damage, gold compounds and methotrexate may cause renal damage, and chloroquine may cause deposits and changes in the cornea of the eye.

The invention is therefore based on the object of providing antiinflammatory agents which have as few side effects as possible.

It has now been found that the effect of known antiinflammatory active ingredients can be considerably enhanced when they are administered in combination with lactalbumin hydrolyzates.

The present invention therefore relates to pharmaceutical compositions based on at least one antiinflammatory active ingredient, which are characterized in that they comprise lactalbumin hydrolyzate or a fraction thereof.

The compositions of the invention provide substantial therapeutic advantages. In particular, substantially lower doses of the antiinflammatory drugs are possible than is conventionally necessary to achieve a particular antiinflammatory effect. It is usually possible to halve or reduce even further the dosage of the antiinflammatory drug while maintaining its main antiinflammatory effect. Fewer side effects attributable to the antiinflammatory drug occur thereby. This is important especially when an antiinflammatory treatment is indicated over a prolonged period, such as, for example, in the treatment of chronic rheumatoid diseases. This is made possible by the enhancement of the antiinflammatory effect of the antiinflammatory drugs by the lactalbumin hydrolyzates, and thus the amount needed to achieve a particular antiinflammatory effect is comparatively lower.

The antiinflammatory drugs and the lactalbumin hydrolyzates can in principle be administered together in one formulation or separately in at least two different formulations. The latter possibility encompasses both simultaneous administration and administration with a time lag, i.e. taking place at different times. Simultaneous administration is preferred, especially in the form of a joint formulation.

Lactalbumins are generally known as constituent of milk and in particular of whey proteins. These are proteins which can be obtained in a manner known per se usually mixed with further milk constituents, i.e. in particular whey proteins such as lactalbumin, lactoferrin, β-lactoglobulin, lysozyme or serum albumin.

The starting material used is fresh raw milk from a domestic animal, preferably cow's milk, which can also have been subjected to a heat treatment which is now conventional in dairies, but is preferably untreated. It can have been skimmed in a conventional way, e.g. by centrifugation.

Removal of caseins can take place in a manner known per se, for example with the aid of so-called rennet or acid precipitation, in which the caseins precipitate while the whey proteins remain in solution. An alternative possibility is to remove the caseins with the aid of a membrane filtration on a microporous membrane with a pore size in the range from 0.1 to 0.6 μm, preferably 0.2 μm. The permeate (or filtrate) contains all the salts, lactose, amino acids, oligopeptides and low molecular weight peptides, while the retentate comprises virtually all the casein constituents of the milk and—unless previously skimmed—also the fat constituents thereof.

Lactalbumins can then be obtained in a manner known per se, in particular by ultrafiltration, from the milk fraction substantially freed of casein and fat. For example, the lactalbumins can be obtained as retentate on a membrane with a cut-off of from 6 to 10 000 Da. It is possible to use for example the product Lactalbumin 75 marketed by Milei.

According to a particular aspect, preference is given according to the invention to the use of lactalbumin hydrolyzates or fractions thereof whose weight average molecular weight is in a range from about 50 to 1200, preferably from about 60 to 800 and in particular from about 80 to 500. Particularly preferred lactalbumin hydrolyzates have a molecular weight distribution with an upper molecular weight limit of about 760 and in particular of 500.

The lactalbumin hydrolyzate to be used according to the invention can be obtained by enzymatic hydrolysis of lactalbumin or a lactalbumin-containing mixture (lactalbumin preparation). For this purpose, an aqueous lactalbumin suspension is treated with at least one protease and, if desired, a lipase.

The hydrolysis generally takes place completely, i.e. until the enzymatic reaction is complete. Depending on the enzyme or enzyme mixture, a degree of hydrolysis of about 20% to about 90%, preferably of about 40% to about 80% and in particular about 75%, can be achieved.

The degree of hydrolysis can be determined and calculated in a manner known per se. It is thus possible to follow the progress of the hydrolysis during the reaction. The formol titration method has proved particularly suitable, according to which the number of free amino groups can be estimated by titration with sodium hydroxide.

The conditions which can be chosen for the purpose of hydrolysis, such as the enzymes employed and the activity thereof, the temperature, the pH, the amount of lactalbumin and the respective concentrations of the reaction mixture, are mutually dependent and can be optimized in the manner of a skilled worker.

The protease(s) used are preferably papain, pancreatin, chymotrypsin and/or trypsin. Optionally, it is also possible to employ proteases obtained from fungi and/or bacteria, especially in addition to the aforementioned proteases. The fungal protease is selected in particular from the proteases from Tritirachium species, in particular Tritirachium alba, proteases from Aspergillus species, in particular Aspergillus saitoi, Aspergillus sojae, Aspergillus oryzae and/or from Rhizopus species, in particular Newlase, and the bacterial protease is selected in particular from proteases from Streptomyces species, in particular Streptomyces caespitosus, Streptomyces griseus (Pronase E.), from Bacillus subtilis types, in particular Subtilopeptidase A (Carlsberg subtilisin), and from Bacillus polymyxa. Further proteases which can be used are proteases obtainable from pineapple, such as bromelain.

If the lactalbumin preparation also contains noticeable quantities of starch and starch-like products, it is advantageous additionally to use a suitable amylase, preferably an α-amylase, e.g. from a subtilis type. The activity optimum of such an amylase is usually at a pH of from 5.7 to 7.2. The reaction temperature can be up to 75° C.

Additional use of a lipase is usually also necessary only if the lactalbumin preparation contains noticeable quantities of fats.

The enzymes are advantageously used in an amount of about 0.01 to 2% by weight based on the suspension.

The protease preferably used is papain or a mixture of approximately equal parts by weight of pancreatin and papain.

A further advantageous protease mixture is a mixture of approximately equal proportions of papain, pancreatin and a bacterial or fungal protease, e.g. the bacterial protease Pronase E from Streptomyces griseus or the fungal protease product “Newlase” from an Aspergillus species. Likewise advantageous is a protease mixture composed of approximately equal proportions of papain, pancreatin and bromelain.

It is particularly advantageous to use, based on approximately 50 kg of lactalbumin as dry matter, approximately 100 to 1000 g, preferably approximately 300 to 700 g and in particular approximately 500 g of papain; approximately 100 to 1000 g, preferably approximately 300 to 700 g and in particular approximately 500 g of pancreatin; and, optionally, approximately 100 to 1000 g, preferably approximately 300 to 700 g, in particular approximately 500 g of a bacterial or fungal protease, in particular Newlase, or of a protease obtainable from pineapple, in particular bromelain.

An approximately 1 to 10% by weight and advantageously a 2 to 5% by weight suspension of lactalbumin in water is expediently used as starting material.

Reaction times in the region of hours are usual, in particular approximately 1 to 8 hours and advantageously approximately 2 to 4 hours; elevated reaction temperatures, approximately 30 to 70° C. and advantageously in the region of the temperature optimum of the enzymes employed; pH values in the slightly acidic to slightly alkaline range, in particular approximately at 6.6 to 7.8 and preferably in the pH optimum of the enzymes employed. On use of enzyme mixtures and especially those with different temperature or pH optima, the respective enzymes can be allowed to act on the lactalbumin at different times by adding part of the enzyme mixture to the reaction mixture at a later time, and adapting the conditions appropriately. In particular, the more thermally stable enzymes can be added after initial, at least partial hydrolysis at moderate temperature employing the less thermally stable enzymes, and subsequently the hydrolysis can be continued using the more thermally stable enzymes at higher temperature. Thus, in the present case, papain and/or pancreatin can be allowed to act on the lactalbumin initially at temperatures in the range from 35 to 38° C. and preferably at approximately 37° C., and subsequently the hydrolysis can be continued through the action of a suitable bacterial and/or fungal protease or of a protease obtainable from pineapple at temperatures in the range of approximately 50 to 75° C. and preferably at approximately 60° C. The procedure for this purpose is expediently such that the protease is added to the reaction mixture and then the temperature is gradually raised. It may be desired in particular for the temperature to be raised gradually to the final temperature during the reaction time which still remains.

Thus, in a particular embodiment of the present invention, there is use of a lactalbumin hydrolyzate which is obtainable by allowing an enzyme mixture containing papain and pancreatin to act on lactalbumin in aqueous suspension at approximately 35 to 38° C. for from 1 to 3 hours; adding a bacterial or fungal protease or a protease obtainable from pineapple, and raising the temperature within from 1 to 4 hours to approximately 60° C., and obtaining the hydrolyzate in a manner known per se.

The procedure for obtaining the hydrolyzate is expediently such that initially the enzymes are inactivated. The reaction mixture can be briefly heated for this purpose. The usual temperatures are in the range from 80 to 85° C., in which case inactivation is achieved after only a few minutes. An alternative possibility is also to inactivate the enzymes by sterilization at ultrahigh temperatures. A few seconds at approximately 130° C. are generally sufficient.

As a consequence, the enzymes usually precipitate and the resulting precipitate is preferably removed after cooling the reaction mixture. For this, it is possible for the reaction mixture to be filtered directly or initially concentrated, for example in vacuo or by spray drying, and for the residue then to be taken up in a suitable solvent and the resulting suspension to be filtered. Solvents suitable in this connection are lower alcohols such as methanol, isopropanol and preferably ethanol or mixtures thereof with water and also organic solvents, especially chloroform.

In this way, for example, approximately 200 mg of lactalbumin hydrolyzate in ethanolic solution are obtained from 5 g of lactalbumin, this hydrolyzate frequently resulting as mixture of the actual lactalbumin peptides as main constituent and further additional constituents. The nature and quantity of the additional constituents, especially fats, depend in particular on the starting material used for the hydrolysis.

In a particular embodiment of the present invention, certain fractions of lactalbumin hydrolyzates which are obtainable in particular from those described above by extraction are used.

It is particularly preferred for the lactalbumin hydrolyzates to be initially defatted before the extraction. Suitable for this purpose are, in a known manner, solvents such as, for example, petroleum ether, which can expediently be employed in a continuous, exhaustive extraction to remove the fat constituents.

Suitable extractants are predominantly polar solvents such as lower alcohols, especially methanol, ethanol, isopropanol and mixtures thereof with water.

Preferred fractions can be obtained by extracting lactalbumin hydrolyzate with absolute ethanol, filtering and obtaining the filtrate. The ethanolic solution contains a lactalbumin hydrolyzate fraction A (ethanol extract) which can be obtained as solid by concentration. This extraction of the preferably defatted lactalbumin hydrolyzate with ethanol preferably takes place by continuous, exhaustive extraction. This also includes removal of nonextractable, i.e. ethanol-insoluble, constituents to obtain an extract solution which can subsequently be concentrated to dryness in order to obtain the lactalbumin hydrolyzate fraction which can be extracted in this way as solid.

Particularly preferred fractions can be obtained by extracting lactalbumin hydrolyzate or fractions thereof with isopropanol, filtering and obtaining the filtrate. The isopropanolic solution contains a lactalbumin hydrolyzate fraction B (isopropanol extract) which can be obtained as solid by concentration. A preferred isopropanol extract can be obtained by employing the residue A obtained by ethanol extraction, and extracting appropriately with isopropanol (lactalbumin hydrolyzate fraction B1).

For this purpose, lactalbumin hydrolyzate and especially the fraction A obtained by the previously described ethanol extraction can be taken up in isopropanol to result in an easily stirrable mixture. For example, the lactalbumin hydrolyzate or a fraction thereof can be stirred with approximately 10 times the volume of isopropanol. The result is usually a suspension which is filtered to remove solids. It has proved to be expedient initially to store the mixture preferably in the cold for some hours up to a few days, and then to filter off the resulting solid phase. About 2 days at a temperature of from 2° C. to 4° C. leads to good results. The filtrate is subsequently concentrated to dryness, it being possible to proceed in accordance with the above statements about the concentration of the ethanol extract. It has additionally proved to be expedient to remove isopropanol residues by repeatedly taking up the residue in ethanol and freeing from solvent by concentration. For example, good results are achieved by taking up in sufficient ethanol and subsequently concentrating three times.

Particularly preferred fractions can be obtained by residue A obtained by ethanol extraction being taken up in absolute ethanol and adjusted with water to an ethanol content of approximately 20 to 60% by volume, preferably of approximtely 30 to 50% by volume and in particular of approximately 40% by volume. The result is usually a suspension, whose solid phase can be removed by filtration. For this, it is expedient for the mixture which is, optionally, initially still in the form of a solution to be stored, preferably in the cold, for several hours to a few days, and then to be filtered. In this case too, 2 days at 2° C. to 4° C. leads to good results. The hydroethanolic solution contains a lactalbumin hydrolyzate fraction C which can be obtained as solid by concentration. Preferred lactalbumin hydrolyzate fractions can be obtained by employing the residue B, and in particular B1, obtained by isopropanol extraction, and proceeding correspondingly, with a lactalbumin hydrolyzate fraction C1 or, in particular, a lactalbumin hydrolyzate fraction C2 being obtained.

The concentration to dryness usually takes place in a manner known per se, e.g. in vacuo or by spray drying. The concentration can expediently take place by evaporation of the ethanol at slightly elevated temperature, for example at approximately 40° C. If desired, the residue obtained is dried further, for example in vacuo. Other, similarly mild methods are likewise suitable. Similar statements apply to the removal of the isopropanol and hydroalcoholic mixtures.

The lactalbumin hydrolyzate fractions are characterized by an increased content of lactalbumin hydrolyzate peptides, a fraction of the initially employed lactalbumin hydrolyzate. According to a particular aspect, this peptide fraction includes 2 peptides (peptide A and peptide B), the proportion of each of which in the peptide fraction is larger than the proportion of any other peptide in this fraction. The two peptides A and B are, in particular, tri- and/or tetrapeptides. According to a further aspect, peptides A and B are characterized by a chromatographic behavior allowing them to be purified by reverse phase chromatography, e.g. on use of an RP 18 column (5 μ material) by elution with an acetonitrile/water/trifluoroacetic acid gradient, with sufficiently different retention times.

Accordingly, peptides A and B can be enriched and obtained from lactalbumin hydrolyzates or fractions thereof.

Particular lactalbumin hydrolyzate peptides of lactalbumin hydrolyzates of the invention are, according to a further aspect, characterized by a molecular ion peak (m+H⁺) in mass spectrometric TOF analysis of m/z=247, 269, 341 and/or 399. The peptides with the molecular ion peaks of 341 and 399 are particularly important. Particular lactalbumin hydrolyzate peptides are therefore to be selected in particular from tripeptides having two proline residues and glutamine or lysine as further amino acid residue and tetrapeptides having a leucine residue or an isoleucine residue, and a proline residue, an alanine residue and a valine residue.

According to one aspect, predominant proportions of lactalbumin hydrolyzates or fractions thereof to be used according to the invention are freely soluble both in chloroform and in water.

These proportions are preferably at least 90% by weight, preferably at least 95% by weight and in particular at least 98% by weight, of the respective lactalbumin hydrolyzate or fraction thereof.

Antiinflammatory drugs in the widest sense include active ingredients which counteract all types of inflammations. These are often divided into non-steroidal antiinflammatory drugs, which essentially intervene in prostaglandin synthesis, and steroidal antiinflammatory drugs, which are commonly also referred to as corticosteroids. Antiinflammatory drugs include in particular antirheumatic drugs.

Antiinflammatory active ingredients which can be particularly used according to the invention are selected from:
a) substances of vegetable origin, frequently in the form of mixtures, e.g. flavones, heparins, terpenes and glycosides, such as infusions, extracts, in particular dry extracts and tinctures of camomile flowers, bromelains, willow bark, arnica, herba symphyti, radix symphyti, Peregrini recentis, horse chestnut seeds;
b) corticoids, in particular mineralocorticoids, such as fludrocortisone and derivatives thereof, optionally in salt form, e.g. fludrocortisone acetate, and glucocorticoids such as dexametasone, hydrocortisone, methylprednisolone, prednisolone, prednisone, triamcinolone, deflazacort, betamethasone, predmylidene, budesonide, chloprednol, fluocortolone, cortisone, rimexolone, flumetasone, flunisolide, fluocortin botyl, fluocinolone, fluocortolone, fluorometholone, fluticasone, beclomethasone and derivatives thereof, optionally in salt form, e.g. dexamethasone sodium 3-sulfobenzoate, dexamethasone sodium phosphate, hydrocortisone sodium succinate, methylprednisolone sodium succinate, prednisolone sodium phosphate, prednisolone acetate, prednisolone sodium succinate, triamcinolone acetonide potassium phosphate, fluticasone 17-propionate, cortisone acetate and beclomesone dipropionate;
c) salicylic acid and derivatives thereof, optionally in salt form, e.g. acetylsalicylic acid, DL-lysine mono(acetylsalicylate);
d) phenylbutazones such as mofebutazone and kebuzone and derivatives thereof, optionally in salt form, e.g. phenylbutazone sodium, mofebutazone sodium and kebuzone sodium;
e) pyrazolones such as phenazone and metamizole and derivatives thereof, optionally in salt form, e.g. metamizole sodium and propyphenazone;
f) paracetamol;
g) anthranilic acid, arylacetic acid and arylpropionic acid derivatives such as acemetazin, ibuprofen, naproxen, diclofenac, indometacin, lonazolac, aceclophenac, fluobiprofen, ketoprofen, naproxen, mefenamic acid, acemetacin, proglumetazin, etofenamate, tiaprofenic acid and dexketoprofen and derivatives thereof, optionally in salt form, e.g. ibuprofen lysine salt, naproxen sodium, diclofenac sodium, lonazolac calcium, ibuprofen sodium dihydrate, proglumetazin dimaleate, dexketoprofen tromethamol and diclofenac cholestyramine;
h) azapropazone, lornoxicam, meloxicam, piroxicam and tenoxicam and derivatives thereof, optionally in salt form;
i) gold compounds, e.g. auranofin and sodium aurothiomalate, sulfur preparations, sulfasalazine, Resochin, chloroquine, dapsone and methotrexate and derivatives thereof, optionally in salt form, e.g. methotrexate sodium, penicillamine, hydroxychloroquine sulfate;
j) mesalazine and cinnarizine, and derivatives thereof, optionally in salt form;
k) dimethyl sulfoxide; in particular
l) selective inhibitors of cyclooxygenase 1, such as diclofenac, ibuprofen, ketoprofen, tiaprofenic acid, indometacin, acemetacin, acetylsalicylic acid, proglumetacin, lonazolac, naproxen, piroxicam, oxyphenbutazone, phenylbutazone, tenoxicam and derivatives thereof, optionally in salt form;
m) selective inhibitors of cyclooxygenase 2, e.g. meloxicam, nabumetone and derivatives thereof, optionally in salt form.

The following antiinflammatory drugs are preferred according to the invention:

prednisone, diclofenac, ibuprofen, indometacin and derivatives thereof, optionally in salt form.

An effective amount of lactalbumin hydrolyzate or of a fraction thereof and an effective amount of at least one antiinflammatory drug, usually formulated in accordance with pharmaceutical or veterinary practice, is administered according to the invention to the individual to be treated, preferably a mammal, especially a human and also an agricultural animal or pet.

Ordinarily, a suitable dose is administered each day, one or more times, optionally together or alternately with other active ingredients or active ingredient-containing products, so that the daily dose administered to an individual to be treated is approximately 2 mg to 100 g, preferably approximately 10 mg to 80 g, advantageously approximately 20 mg to 60 g, and in particular approximately 50 mg to 60 g, of lactalbumin hydrolyzate on oral administration, approximately 0.5 mg to 80 g, preferably approximately 8 mg to 70 g, advantageously approximately 20 mg to 70 g, and in particular approximately 30 mg to 40 g, of lactalbumin hydrolyzate on parenteral administration and approximately 0.5 mg to 100 g, preferably approximately 1 mg to 80 g, advantageously approximately 5 mg to 60 g, and in particular approximately 10 mg to 60 g, of lactalbumin hydrolyzate on topical use. The amounts of lactalbumin fractions to be administered are usually below the aforementioned dosages. Thus, the dosage for the lactalbumin hydrolyzate constituents may be chosen to be approximately a factor of 5 to 20 and the dosage for the lactalbumin hydrolyzate peptides may be chosen to be approximately a factor of 10 to 30 lower.

It is moreover possible to choose the dosage of the antiinflammatory drug according to the invention to be lower than conventionally recommended for achieving the main antiinflammatory effect. Accordingly, the daily dose recommended in particular for the treatment of rheumatoid diseases can be reduced by at least a factor of 2 and advantageously by at least a factor of 5. If, for example, 40 mg of prednisone are administered each day to an asthmatic during an attack in order to keep his asthma compensated, it is possible according to the invention to achieve the same therapeutic effect with a combination of 10 mg of lactalbumin hydrolyzate fraction C1 or C2 and only 7.5 mg of prednisone. Correspondingly, a daily dose of 20 mg of prednisone could be reduced to only 3.5 to 4 mg of prednisone by combined administration of 10 mg of lactalbumin hydrolyzate fraction C1 or C2.

The extent to which the dose of an active ingredient acknowledged to have an antiinflammatory effect can be reduced when it is used according to the invention in combination with lactalbumin hydrolyzate or a fraction thereof can be demonstrated in particular by controlled, randomized double-blind studies. It is possible to use for this purpose for example clinical variables which document in accordance with scientific knowledge the progress of the therapy of the pathological state investigated. Animal models can also be used in addition.

For example, on use according to the invention of a combination of diclofenac and a lactalbumin hydrolyzate fraction on the one hand and of a 10-times higher diclofenac dose without use of a lactalbumin hydrolyzate fraction of the invention for the treatment of seropositive rheumatoid arthritis (RA), the therapy is assessed by biochemical monitoring of the changes in the CRP levels (C-reactive protein), the erythrocyte sedimentation rate (ESR) and recording of the pain experienced by means of scores. The degree of reduction in the dose is ascertained by referring to reference values for the therapy of RA.

The amounts and proportions of active ingredients are based on the active active ingredient, so that an appropriate recalculation is necessary for salts and derivatives.

The compositions of the invention are particularly suitable for the treatment of all types of inflammation. The present invention therefore also relates to the use of lactalbumin hydrolyzates or fractions thereof in combination with at least one antiinflammatory drug for the treatment of all types of inflammation.

According to a particular aspect, it is possible to treat rheumatoid diseases. These include, in particular, disorders of the locomotory systems such as signs of wear, degenerative processes and inflammatory states of the joints, spine, muscles and tendons, e.g. acute affections of the locomotor system such as bursitis, tendinitis, synovitis, tendovaginitis, periarthritis of the shoulder, lumbagos, sprains and strains, painful swellings or inflammations after injuries or operations, rheumatic complaints such as arthroses, irritation associated with arthroses and spondylarthroses, acute and chronic arthritides, polyarthritis, rheumatoid arthritis, gout, Bechterew's disease, soft tissue rheumatism, superficial thrombophlebitis.

A particular aspect of a treatment in the sense according to the invention relates to the treatment of chronic disorders.

The invention also relates to the production of compositions for the treatment of an individual, preferably of a mammal, in particular of a human and of an agricultural animal or pet.

Compositions of the invention are usually based on an active ingredient combination and, optionally, a formulation base.

The formulation base of formulations of the invention comprises physiologically acceptable excipients. Physiologically acceptable excipients are those known to be usable in the pharmacy sector, in food technology and adjacent areas, in particular those listed in relevant pharmacopeias (e.g. DAB, Ph. Eur., BP, NF), and also other excipients whose properties do not preclude physiological use.

Suitable excipients may be: wetting agents; emulsifying and suspending agents; preservatives; antioxidants; antiirritants; chelating agents; tablet-coating aids; emulsion stabilizers; film formers; gel formers; masking odors; masking flavors; resins; hydrocolloids; solvents; solubilizers; neutralizers; permeation enhancers; pigments; quaternary ammonium compounds; refatting and superfatting agents; ointment, cream or oil bases; silicone derivatives; spreading aids; stabilizers; sterilants; suppository bases; tablet excipients such as binders, fillers, lubricants, disintegrants or coatings; propellants; desiccants; opacifiers; thickeners; waxes; plasticizers; white oils. An arrangement concerning this is based on expert knowledge as set forth for example in Fiedler, H. P., Lexikon der Hilfsstoffe für Pharmazie, Kosmetik und angrenzende Gebiete, 4th edition, Aulendorf: ECV-Editio-Cantor-Verlag, 1996.

Examples of suitable pharmaceutical formulations are solid pharmaceutical forms such as oral powders, dusting powders, granules, tablets, especially film-coated tablets, pastilles, sachets, cachets, sugar-coated tablets, capsules such as hard and soft gelatin capsules, suppositories or vaginal pharmaceutical forms, semisolid pharmaceutical forms such as ointments, creams, hydrogels, pastes or patches, and liquid pharmaceutical forms such as solutions, emulsions, especially oil-in-water emulsions, suspensions, for example lotions, preparations for injection and infusion, eye drops. Implanted delivery devices can also be used to administer active ingredients of the invention. A further possibility is also to use liposomes or microspheres.

The formulations may be administered for example by the oral, rectal, topical, in particular transdermal, parenteral, in particular subcutaneous, intravenous, intramuscular, intraocular or intranasal route. Preference is given to oral, topical and, if necessary, also intravenous administration.

The formulations are produced usually by mixing or diluting the active ingredients with a suitable excipient. Excipients may be solid, semisolid or liquid materials which serve as vehicle, carrier or medium for the active ingredient. Admixture of further excipients, if necessary, takes place in a manner known per se. It is possible to carry out shaping steps, optionally in conjunction with mixing processes, e.g. a granulation, compression and the like.

It is possible in particular for the active ingredient components to be formulated together. However, they can also be initially processed separately and then be combined in a compartmented, e.g. multilayer, pharmaceutical form. It is possible in this way to take account of possible active ingredient incompatibilities and different active ingredient properties such as bioavailability, stability, solubility and the like.

Besides combination products, the invention also relates to corresponding single-drug products in the form of commercial packs from which the combined use of the invention is to be taken.

The present invention is explained in detail by means of the following examples, without being restricted thereto.

Preparation of Lactalbumin Hydrolyzate and Fractions Thereof

### EXAMPLE 1

Preparation of a Lactalbumin Hydrolyzate

5 g of lactalbumin are suspended in 130 ml of water, 50 mg of papain and 50 mg of pancreatin are added, the mixture is heated to 35-37° C. and stirred at this temperature for about 2 hours, 50 mg of a commercially available fungal protease, e.g. Newlase, a Rhizopus protease with an activity of approximately 0.5 U/mg of mass, are added, and the temperature is slowly (over the course of 2-3 hours) raised to 60° C. The temperature is then increased briefly to 80° C., the cloudy solution is allowed to cool and is concentrated in vacuo, and the residue is taken up in ethanol, filtered and concentrated in vacuo. About 0.2 g of lactalbumin hydrolyzate is obtained.

### EXAMPLE 2

Defatting of the Lactalbumin Hydrolyzate

100 g of the lactalbumin hydrolyzate obtained in example 1 are defatted in a continuous, exhaustive reaction with 2 l of petroleum ether at a temperature of approximately 22° C.

### EXAMPLE 3

Obtaining a Lactalbumin Hydrolyzate Fraction A

5 g of the defatted lactalbumin hydrolyzate obtained in example 2 are extracted with 100 ml of absolute ethanol at a temperature of 22° C. in a continuous, exhaustive reaction. The ethanol extract is evaporated at 40° C. and dried in vacuo.

### EXAMPLE 4

Obtaining a Lactalbumin Hydrolyzate Fraction B

500 g of the lactalbumin hydrolyzate fraction A obtained in example 3 are stirred with 10 times the volume of isopropanol and stored at a temperature of 2° C. to 4° C. for 2 days. The clear solution from filtration or centrifugation is concentrated to dryness in vacuo at 50° C.

Isopropanol residues are removed by taking up the residue in 2 l of ethanol and evaporating the ethanol at 40° C. in vacuo. This procedure is repeated 2-3 times. About 400 g of a lactalbumin hydrolyzate fraction B1 are obtained as a yellowish powder.

### EXAMPLE 5

Obtaining a Lactalbumin Hydrolyzate Fraction C

The lactalbumin hydrolyzate fraction B1 freed of isopropanol residues in example 4 is dissolved in 2 l of absolute ethanol, and the ethanol content is adjusted to 40% by volume by adding water. The solution is then stored at 2-4° C. for 2 days. The clear solution resulting from filtration or centrifugation is concentrated to dryness at 40° C. in vacuo. About 300 g of a lactalbumin hydrolyzate fraction C2 are obtained as a yellowish powder.

Mass spectrometic analysis of the lactalbumin hydrolyzate fraction C2 reveals the following peaks (m/z+/−0.5; TOF MS ES+):
72.09; 86.10; 98.99; 120.09; 127.08; 149.03; 185.18; 188.08; 199.19; 229.17; 244.18; 247.12; 269.17; 286.99; 288.98; 341.23; 349.01; 360.21; 399.27; 431.25; 453.23; 469.23; 496.35; 525.36; 527.37; 557.35; 565.28; 569.30; 587.26; 587.28; 609.38; 649.35; 654.41; 682.42; 683.36; 723.37; 724.36; 763.40; 764.43; 772.42; 773.44; 797.42; 837.47; 838.49; 860.45; 871.52; 872.51; 889.54; 926.55; 927.51; 948.52; 949.46; 951.53.

Investigation of the lactalbumin hydrolyzate fraction C2 by HPLC analysis (reverse phase RP-18; 5 μ material; elution with an acetonitrile/water/trifluoroacetic acid gradient; detection at 205 nm) shows besides free amino acids a peptide mixture which, in the oligopeptide region (dipeptides to decapeptides), comprises two main fractions which, relative to the hydrophobic amino acids which elute at about 7.5 min, especially tryptophan, elute at about 16 min and about 19.5 min, respectively. These main fractions are, according to their chromatographic behavior, tri- or tetrapeptides, so that it is to be assumed that a substantial constituent of the lactalbumin hydrolyzates of the invention is formed by two peptides, probably tri- and/or tetrapeptides.

These peptides can be isolated by an appropriate preparative HPLC.

Pharmaceutical Compositions

### Example 6

| a) Soft gelatin capsule with diclofenac | | |
|---|---|---|
| Filling: | | |
| Diclofenac | 15 | mg |
| Lactalbumin hydrolyzate fraction from Ex. 5 | 15 | mg |
| Soybean oil (ref.) | 440 | mg |
| Soybean lecithin (E322) | 50 | mg |
| Colloidal silica | 5 | mg |
| Capsule shell: | | |
| Gelatin | 303 | mg |
| Glycerol 85% | 87 | mg |
| Sorbitol 70% | 77 | mg |
| Purified water | 52 | mg |
| Iron oxide pigment Braun 75 (E 172) | 3 | mg |

1 to 2 Capsules Once a Day are to be Recommended for the Treatment of RA

| b) Tablet with ibuprofen | | |
|---|---|---|
| Ibuprofen | 40 | mg |
| Lactalbumin hydrolyzate fraction from Ex. 5 | 20 | mg |
| Lactose | 127.5 | mg |
| Magnesium stearate | 5 | mg |
| Talc | 23.75 | mg |
| Microcrystalline cellulose | 81 | mg |

1 to 2 tablets a day are to be recommended for the treatment of rheumatoid diseases.
c) Tablets produced as in b) can moreover be provided in a known manner with a film coating which is soluble in the stomach or small intestine.

### EXAMPLE 7

Ibuprofen Study:

Five patients with ulcerative colitis (Crohn's disease) who regularly took between 1800 mg and 2400 mg of ibuprofen were treated twice a day with 50 mg of ibuprofen and 10 mg of lactalbumin hydrolyzate fraction from example 5. The result of treatment was assessed on the basis of the CRP level, the ESR and the pain experienced in scores. Control was successful in all 5 cases.

No improvement was detectable if only the lactalbumin hydrolyzate fraction, but not the ibuprofen, was administered to the patients; on the contrary, the findings deteriorated as if without therapy. On treatment of the same patients subsequently with the combination of the invention composed of 500 mg of ibuprofen and 10 mg of lactalbumin hydrolyzate fraction twice a day each, an improvement in the symptoms was detectable within a maximum of 4 days.

### EXAMPLE 8

Diclofenac Study:

Four patients with seropositive arthritis (RA) who regularly took between 100 mg and 150 mg of diclofenac were treated with a combination of 10 mg of diclofenac and 10 mg of lactalbumin hydrolyzate fraction from example 5. The result of treatment was assessed on the basis of the CRP level, the ESR and the pain experienced in scores. The measurements found during the combination therapy of the invention corresponded in one case to the values which the patient showed on treatment with 100 mg of diclofenac, and in three cases corresponded to the values which the patients showed on treatment with 150 mg of diclofenac.

On administration only of the lactalbumin hydrolyzate fraction, but not of the diclofenac, to the patients, in all cases the symptoms reappeared in their entirety after an average of 6.5 days. On renewed therapy with 10 mg of diclofenac and 10 mg of lactalbumin hydrolyzate fraction there was found to be within 2.5 days on average in all cases a distinct improvement in the clinical picture and symptoms.

### EXAMPLE 9

Prednisone Study:

Nine patients with allergic bronchial asthma to whom a daily dose between 20 mg and 40 mg of prednisone was regularly administered were treated according to the invention each day with a combination of the invention composed of 5 mg of prednisone and 10 mg lactalbumin hydrolyzate fraction from example 5.

In 8 cases, the patients were free of symptoms within a short time. In one case, the prednisone dose had to be reduced to 20 mg a day. This patient had previously been treated with a daily dose of 40 mg.

If only the lactalbumin hydrolyzate fraction, but not the prednisone, was administered to the patients, this therapy had to be terminated after 2.5 days on average, because the asthma symptoms in their entirety, like those in patients without therapy, recurred. Renewed therapy with 5 mg of prednisone and 10 mg of lactalbumin hydrolyzate fraction led to an improvement in the symptoms within 2 days.

1. A pharmaceutical composition comprising:
at least one antiinflammatory active ingredient,
wherein the pharmaceutical composition comprises lactalbumin hydrolyzate or a fraction thereof.

2. The pharmaceutical composition as claimed in claim 1, wherein the lactalbumin hydrolyzate or the fraction thereof has a weight average molecular weight of about 50 to 1200.

3. The pharmaceutical composition as claimed in claim 1, wherein the lactalbumin hydrolyzate is obtained by enzymatic hydrolysis with papain, pancreatin and at least one bacterial protease.

4. The pharmaceutical composition as claimed in claim 1, wherein the lactalbumin hydrolyzate fraction is obtained by a process which comprises:
extracting a lactalbumin hydrolyzate with ethanol and
obtaining the ethanol extract, if desired as dry extract.

5. The pharmaceutical composition as claimed in claim 1, wherein the lactalbumin hydrolyzate fraction is obtained by a process which comprises
extracting a lactalbumin hydrolyzate with ethanol,
concentrating the extract to dryness,
extracting the resulting ethanol dry extract with isopropanol and
obtaining the isopropanol extract, if desired as dry extract.

6. The pharmaceutical composition as claimed in claim 1, wherein the lactalbumin hydrolyzate fraction is obtained by a process which comprises
extracting a lactalbumin hydrolyzate with ethanol,
concentrating extract to dryness,
extracting the resulting ethanol dry extract with isopropanol,
concentrating the extract to dryness,
taking up the resulting isopropanol dry extract in ethanol,
adding water until the ethanol:water ratio is about 20:80 to 60:40,
removing the precipitates, and
obtaining the hydroethanolic extract, if desired as dry extract.

7. The pharmaceutical composition as claimed in claim 1, wherein the antiinflammatory ingredient is selected from the group consisting of extracts and tinctures of camomile flowers, bromelains, willow bark, arnica, herba symphyti, radix symphyti, Peregrini recentis and horse chestnut seeds, fludrocortisone, dexametasone, hydrocortisone, methylprednisolone, prednisolone, prednisone, triamcinolone, deflazacort, betamethasone, predmylidene, budesonide, chloprednol, fluorocortolone, cortisone, rimexolone, flumetasone, flunisolide, fluocortin botyl, fluocinolone, fluocortolone, fluorometholone, fluticasone, beclomethasone, salicylic acid, mofebutazone, kebuzone, phenazone, metamizole, paracetamol, acemetazin, ibuprofen, naproxen, diclofenac, indometacin, lonazolac, aceclophenac, fluobiprofen, ketoprofen, naproxen, mefenamic acid, acemetacin, proglumetazin, etofenamate, tiaprofenic acid, dexketoprofen, azapropazone, lornoxicam, meloxicam, piroxicam, tenoxicam, auranofin and sodium aurothiomalate, sulfasalazine, Resochin, chloroquine, dapsone, methotrexate, mesalazine, cinnarizine, dimethyl sulfoxide, derivatives thereof, salts thereof, and mixtures thereof.

8. The pharmaceutical composition as claimed in claim 7, wherein the antiinflammatory active ingredient is selected from the group consisting of prednisone, diclofenac, ibuprofen, indometacin, derivatives thereof, salts thereof, and mixtures thereof.

9. A method for treating rheumatoid diseases, which comprises:
administering to a mammal in need thereof an effective amount of the pharmaceutical composition as claimed in claim 1.

10. A method for the treatment of rheumatoid diseases, where at least one antiinflammatory active ingredient is administered in combination with lactalbumin hydrolyzate or a fraction thereof to a patient.
